(19) United States
(12) Patent Application Publication
(10) Pub. No.: US 2023/0256223 A1
(43) Pub. Date: Aug. 17, 2023

(54) Utilizing Ultrasound Waves for Infection Prevention in Vascular Access Devices

(71) Applicant: Becton, Dickinson and Company, Franklin Lakes, NJ (US)

(72) Inventors: Amir Harandi, Bedminster, NJ (US); Eric Dustin Agdeppa, Cary, NC (US); Sandeep Tripathi, New York, NY (US); Jack Balji, Mahwah, NJ (US); Shishir Prasad, Ramsey, NJ (US)

(21) Appl. No.: 18/101,844

(22) Filed: Jan. 26, 2023

Related U.S. Application Data
(60) Provisional application No. 63/303,814, filed on Jan. 27, 2022.
(60) Provisional application No. 63/305,466, filed on Feb. 1, 2022.

(51) Int. Cl. A61M 39/02

(52) U.S. Cl. CPC A61M 39/0247; A61M 2039/0258; A61M 2039/0261; A61M 2039/0285

(57) ABSTRACT

A method of preventing infections in vascular access devices in which an acoustic wave generator is attached to the vascular access catheter, and the acoustic wave generator is activated to produce acoustic waves, which are transmitted to the vascular access catheter to create mechanical vibrations in the vascular access catheter. The acoustic waves may be ultrasound waves. The acoustic wave generator may be attached to a catheter tube of the vascular access catheter or attached to a hub of the vascular access catheter. The acoustic wave generator a separate device that is removably clipped to the vascular access catheter, may be provided in a vascular access catheter stabilization device or may be integral with the vascular access catheter.

### CROSS-REFERENCE TO RELATED APPLICATIONS

The present application claims priority to U.S. Provisional Application Serial No. 63/303,814, filed Jan. 27, 2022 and U.S. Provisional Application Serial No. 63/305,466, filed Feb. 1, 2022, the entire disclosures of each of which are incorporated by reference in their entireties.

### BACKGROUND OF THE INVENTION

### Field of the Invention

The present invention is directed to a method for preventing infections in vascular access catheters, and more specifically, a method of preventing infections in vascular access catheters by transmitting acoustic waves to the vascular access catheter, thereby generating mechanical vibrations in the vascular access catheter which reduce or prevent the build-up of microbial biofilms on the walls of the vascular access catheter.

### Description of Related Art

The placement of intravenous (IV) catheters into a patient’s blood vessel to administer fluids and medications directly into a patient’s bloodstream is one of the most common invasive hospital procedures performed worldwide. However, even the most rigorously performed studies indicate that the overall IV catheter failure rate lies between 35% and 50%. Failures may take a variety of forms, including infection, any of which alone, or in combination, lead to removal of the catheter before the end of its intended dwell time.

The formation and build-up of microbial biofilms, which often are formed by antimicrobial-resistant organisms, on the walls of the vascular access catheter are responsible for 65% of the vascular access catheter related infections treated in the developed world. The presence of microbial biofilms in a vascular access catheter can result in complications ranging from removing the vascular access catheter from the patient and inserting a new one to the patient contracting a serious biofilm related disease.

There are various methods to avoid or reduce microbial biofilm growth in IV catheters including routine chemical cleaning and disinfecting caps provided on the catheter when it is not being used. However, some of these methods are invasive and/or only moderately effective.

It has been found that mechanical vibration energy is effective to inhibit the adhesion of microbial biofilms to the vascular access catheter walls. However, there is a need for a convenient way to apply the mechanical vibration energy intermittently or continuously to the vascular access catheter after the catheter has been inserted in the patient’s blood vessel.

### SUMMARY OF THE INVENTION

The present invention is directed to a vascular access catheter stabilization device comprising a hub engagement portion and a base adapted for removable attachment to a patient’s skin. The hub engagement portion comprises a housing enclosing an acoustic wave generator and is adapted to additionally surround at least a portion of a hub of the vascular access catheter.

The acoustic wave generator may comprise a driver electrically connected to a piezoelectric plate. The driver may comprise a power source for supplying electric current and a control unit for controlling the transmission of the electric current to the piezoelectric plate. The piezoelectric plate is positioned within the housing to be in contact with the hub of the vascular access catheter when the hub of the vascular access catheter is enclosed in the housing and may have a size and shape corresponding to an upper surface of at least a section of the portion of the hub of the vascular access catheter enclosed within the housing, such that contact between the piezoelectric plate and the hub of the vascular access catheter is sufficient to allow transmission of acoustic waves generated in the piezoelectric plate to the hub of the vascular access catheter. The acoustic waves generated by the acoustic wave generator may have a frequency in the ultrasonic range above 20 kHz.

The vascular access catheter stabilization device may further comprise a compressible cushioning spacer that acts to fill any gaps within the housing and assure that the piezoelectric plate positively contacts the hub of the vascular access catheter. The cushioning spacer may be positioned between the driver and the piezoelectric plate.

The housing may comprise a top wall, a bottom wall, a proximal wall, a distal wall, and two side walls, where the proximal wall and the distal wall each have an opening to allow the vascular access catheter to pass through the housing when the hub of the vascular access catheter is at least partially surrounded by the housing. The driver, the piezoelectric plate, and the cushioning spacer may be contained in an upper portion of the housing between the top wall and the hub of the vascular access catheter. The bottom wall may have a recess that is sized and shaped to receive at least a portion of the hub of the vascular access catheter. The recess may comprise areas that receive wings extending from a shaft portion of the hub of the vascular access catheter and surround edges of the wings to stabilize the hub of the vascular access catheter within the housing.

The housing may comprise two parts, a first part including the top wall and a second part including the bottom wall, where the first part of the housing is removably connected to or locked to the second part of the housing.

A connection between the first part of the housing and the second part of the housing may comprise one or more protrusions on the first part of the housing or the second part of the housing that are received within corresponding openings in the other of the first part of the housing and the second part of the housing. The one or more protrusions may comprise a flexible beam attached to the housing at one end and, at the other end, having a tab having a bottom surface extending substantially perpendicularly from the flexible beam and an angled top surface.

Flanges may extend substantially perpendicularly from each side wall of each of the first part of the housing and the second part of the housing and the connection or locking of the first part of the housing to the second part of the housing may be provided via the flanges.

The base may be an adhesive pad.

The present invention is also directed to a method of reducing build-up of microbial biofilms in a vascular access catheter. In the inventive method, a vascular access catheter is inserted into the blood vessel of a patient, the hub of the vascular access catheter is placed into the housing of the vascular access catheter stabilization device described above that has been secured by the base to the patient’s skin, the acoustic wave generator is activated, and the acoustic waves are transmitted to the vascular access catheter creating mechanical vibrations in the catheter.

When the vascular access catheter stabilization device has a two-part housing, the method may further include placing the hub of the vascular access catheter into the second part of the housing of the vascular access catheter stabilization device which has been secured by the base to the patient’s skin, placing the first part of the housing of the vascular access catheter stabilization device over the hub of the vascular access catheter, and connecting the first part of the housing of the vascular access catheter stabilization device to the second part of the housing of the vascular access catheter stabilization device.

The present invention is also directed to a vascular access catheter comprising a catheter tube, a hub comprising a housing through which the catheter tube passes, and an acoustic wave generator enclosed within the housing. Acoustic waves generated by the acoustic wave generator may be transmitted to the catheter tube via the hub or may be transmitted directly to the catheter tube to create mechanical vibrations in the catheter tube.

The acoustic wave generator may comprise a driver electrically connected to a piezoelectric plate. The driver may comprise a power source for supplying electric current and a control unit for controlling the transmission of the electric current to piezoelectric plate. The acoustic waves generated by the acoustic wave generator may have a frequency in the ultrasonic range above 20 kHz.

The vascular access catheter may further comprise a compressible cushioning spacer that acts to fill any gaps within the housing and assure that the piezoelectric plate positively contacts the hub or the catheter tube. The cushioning spacer may be positioned between the driver and the piezoelectric plate.

The hub may include wings extending substantially perpendicularly from opposite sides of the housing.

The present invention is also directed to a method of reducing build-up of microbial biofilms in a vascular access catheter. In the inventive method, the vascular access catheter described above is inserted into the blood vessel of a patient, the acoustic wave generator is activated, and the acoustic waves are transmitted to the vascular access catheter creating mechanical vibrations in the catheter.

In a further configuration, the present invention is directed to a method of preventing infections in vascular access devices. In the method, an acoustic wave generator is attached to the vascular access catheter. The acoustic wave generator is then activated to produce acoustic waves, which are transmitted to the vascular access catheter to create mechanical vibrations in the vascular access catheter. The acoustic waves generated by the acoustic wave generator may have a frequency in the ultrasonic range. The acoustic wave generator may be attached to a catheter tube of the vascular access catheter or attached to a hub of the vascular access catheter. The acoustic wave generator may have a contact area having a size and shape corresponding to a surface of the vascular access catheter, such that contact between the acoustic wave generator and the vascular access catheter is sufficient to allow transmission of the acoustic waves generated by the acoustic wave generator to the vascular access catheter.

The acoustic wave generator may comprise a housing enclosing a driver electrically connected to a piezoelectric plate. The driver may comprise a power source for supplying electric current and a control unit for controlling the transmission of the electric current to piezoelectric plate. The acoustic wave generator may further comprise a compressible cushioning spacer that acts to fill any gaps within the housing. The cushioning spacer may be positioned between the driver and the piezoelectric plate.

The acoustic wave generator may be a separate device that is removably clipped to the vascular access catheter.

Alternatively, the acoustic wave generator may be provided in a vascular access catheter stabilization device comprising a hub engagement portion comprising a housing enclosing the acoustic wave generator and adapted to additionally surround at least a portion of a hub of a vascular access catheter, and a base adapted for removable attachment to a patient’s skin. The base is an adhesive pad.

The method may further comprise inserting the vascular access catheter into the blood vessel of a patient and placing the hub of the vascular access catheter into the housing of the vascular access catheter stabilization device, which has been secured by the base to the patient’s skin, prior to activating the acoustic wave generator.

In another alternative, the acoustic wave generator may be integral with the vascular access catheter, where the vascular access catheter comprises a hub comprising a housing through which a catheter tube passes, and the acoustic wave generator may be enclosed within the housing. The acoustic waves generated by the acoustic wave generator may be transmitted to the catheter tube via the hub to create mechanical vibrations in the catheter tube or the acoustic waves generated by the acoustic wave generator may be transmitted directly to the catheter tube.

The method may further comprise inserting the catheter tube of the vascular access catheter into the blood vessel of a patient prior to activating the acoustic wave generator.

The vascular access catheter stabilization device may comprise a hub engagement portion and a base adapted for removable attachment to a patient’s skin. The hub engagement portion may comprise a housing enclosing an acoustic wave generator and is adapted to additionally surround at least a portion of a hub of the vascular access catheter.

The acoustic wave generator may comprise a driver electrically connected to a piezoelectric plate. The driver may comprise a power source for supplying electric current and a control unit for controlling the transmission of the electric current to the piezoelectric plate. The piezoelectric plate is positioned within the housing to be in contact with the hub of the vascular access catheter when the hub of the vascular access catheter is enclosed in the housing and may have a size and shape corresponding to an upper surface of at least a section of the portion of the hub of the vascular access catheter enclosed within the housing, such that contact between the piezoelectric plate and the hub of the vascular access catheter is sufficient to allow transmission of acoustic waves generated in the piezoelectric plate to the hub of the vascular access catheter. The acoustic waves generated by the acoustic wave generator may have a frequency in the ultrasonic range above 20 kHz.

The vascular access catheter stabilization device may further comprise a compressible cushioning spacer that acts to fill any gaps within the housing and assure that the piezoelectric plate positively contacts the hub of the vascular access catheter. The cushioning spacer may be positioned between the driver and the piezoelectric plate.

The housing may comprise a top wall, a bottom wall, a proximal wall, a distal wall, and two side walls, where the proximal wall and the distal wall each have an opening to allow the vascular access catheter to pass through the housing when the hub of the vascular access catheter is at least partially surrounded by the housing. The driver, the piezoelectric plate, and the cushioning spacer may be contained in an upper portion of the housing between the top wall and the hub of the vascular access catheter. The bottom wall may have a recess that is sized and shaped to receive at least a portion of the hub of the vascular access catheter. The recess may comprise areas that receive wings extending from a shaft portion of the hub of the vascular access catheter and surround edges of the wings to stabilize the hub of the vascular access catheter within the housing.

The housing may comprise two parts, a first part including the top wall and a second part including the bottom wall, where the first part of the housing is removably connected to or locked to the second part of the housing.

A connection between the first part of the housing and the second part of the housing may comprise one or more protrusions on the first part of the housing or the second part of the housing that are received within corresponding openings in the other of the first part of the housing and the second part of the housing. The one or more protrusions may comprise a flexible beam attached to the housing at one end and, at the other end, having a tab having a bottom surface extending substantially perpendicularly from the flexible beam and an angled top surface.

Flanges may extend substantially perpendicularly from each side wall of each of the first part of the housing and the second part of the housing and the connection or locking of the first part of the housing to the second part of the housing may be provided via the flanges.

The base may be an adhesive pad.

The present invention is also directed to a method of reducing build-up of microbial biofilms in a vascular access catheter. In the inventive method, a vascular access catheter is inserted into the blood vessel of a patient, the hub of the vascular access catheter is placed into the housing of the vascular access catheter stabilization device described above that has been secured by the base to the patient’s skin, the acoustic wave generator is activated, and the acoustic waves are transmitted to the vascular access catheter creating mechanical vibrations in the catheter.

When the vascular access catheter stabilization device has a two-part housing, the method may further include placing the hub of the vascular access catheter into the second part of the housing of the vascular access catheter stabilization device which has been secured by the base to the patient’s skin, placing the first part of the housing of the vascular access catheter stabilization device over the hub of the vascular access catheter, and connecting the first part of the housing of the vascular access catheter stabilization device to the second part of the housing of the vascular access catheter stabilization device.

The vascular access catheter may comprise a catheter tube, a hub comprising a housing through which the catheter tube passes, and an acoustic wave generator enclosed within the housing. Acoustic waves generated by the acoustic wave generator may be transmitted to the catheter tube via the hub or may be transmitted directly to the catheter tube to create mechanical vibrations in the catheter tube.

The acoustic wave generator may comprise a driver electrically connected to a piezoelectric plate. The driver may comprise a power source for supplying electric current and a control unit for controlling the transmission of the electric current to piezoelectric plate. The acoustic waves generated by the acoustic wave generator may have a frequency in the ultrasonic range above 20 kHz.

The vascular access catheter may further comprise a compressible cushioning spacer that acts to fill any gaps within the housing and assure that the piezoelectric plate positively contacts the hub or the catheter tube. The cushioning spacer may be positioned between the driver and the piezoelectric plate.

The hub may include wings extending substantially perpendicularly from opposite sides of the housing.

The present invention is also directed to a method of reducing build-up of microbial biofilms in a vascular access catheter. In the inventive method, the vascular access catheter described above is inserted into the blood vessel of a patient, the acoustic wave generator is activated, and the acoustic waves are transmitted to the vascular access catheter creating mechanical vibrations in the catheter.

### BRIEF DESCRIPTION OF THE DRAWINGS

FIG. 1 is a top, side perspective view of a vascular access catheter stabilization device according to the invention in use;

FIG. 2 is an exploded top, side perspective view of the vascular access catheter stabilization device of FIG. 1;

FIG. 3 is an enlarged top, side perspective view of the portion within the dotted lines in FIG. 1; and

FIG. 4 is a cross-section schematic view of an acoustic wave generator attached to the catheter tube of a vascular access catheter.

### DESCRIPTION OF THE INVENTION

As used herein, any numerical values are expressed using a period as a decimal point and a comma as a thousand separator, for example, 1,234 would be one thousand two hundred thirty four, and 1.2 would be one and two tenths. Unless otherwise expressly specified, all numbers such as those expressing values, ranges, amounts or percentages may be read as if prefaced by the word “about”, even if the term does not expressly appear. Any numerical range recited herein is intended to include all sub-ranges subsumed therein. For example, a range of “1 to 10” is intended to include any and all sub-ranges between and including the recited minimum value of 1 and the recited maximum value of 10, that is, all subranges beginning with a minimum value equal to or greater than 1 and ending with a maximum value equal to or less than 10, and all subranges in between, e.g., 1 to 6.3, or 5.5 to 10, or 2.7 to 6.1. Plural encompasses singular and vice versa. When ranges are given, any endpoints of those ranges and/or numbers within those ranges can be combined with the scope of the present invention. “Including”, “such as”, “for example” and like terms means “including/such as/for example but not limited to”.

For purposes of the description hereinafter, spatial orientation terms, as used, shall relate to the referenced embodiment as it is oriented in the accompanying drawings, figures, or otherwise described in the following detailed description. However, it is to be understood that the embodiments described hereinafter may assume many alternative variations and configurations. It is also to be understood that the specific components, devices, features, and operational sequences illustrated in the accompanying drawings, figures, or otherwise described herein are simply exemplary and should not be considered as limiting.

As used herein, the terms “vascular access catheter” refers to any vascular access device including intravenous (IV) catheters. Vascular access catheters include, but are not limited to, peripheral intravenous catheter (PIV), peripherally inserted central catheters (PICC), centrally inserted central catheters (CICC), midline peripheral catheters, central venous catheters (CVC), central venous catheters (CVC) and implanted venous ports.

The present invention is directed to a method of preventing infections in vascular access catheters utilizing acoustic waves. An acoustic wave generator produces acoustic waves that are transferred to the vascular access catheter, thereby generating mechanical vibrations in the vascular access catheter. The mechanical vibration reduce or prevent the build-up of microbial biofilms on the walls of the vascular access catheter, thereby preventing infections.

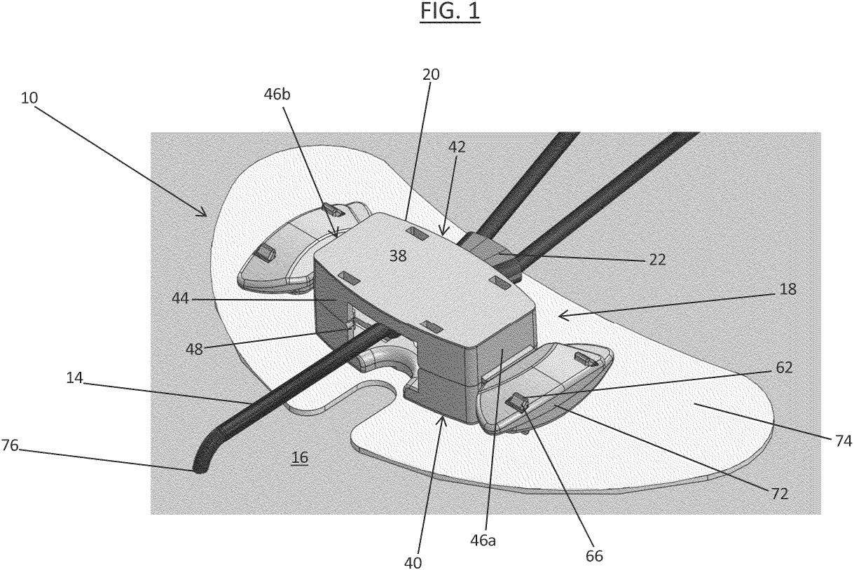

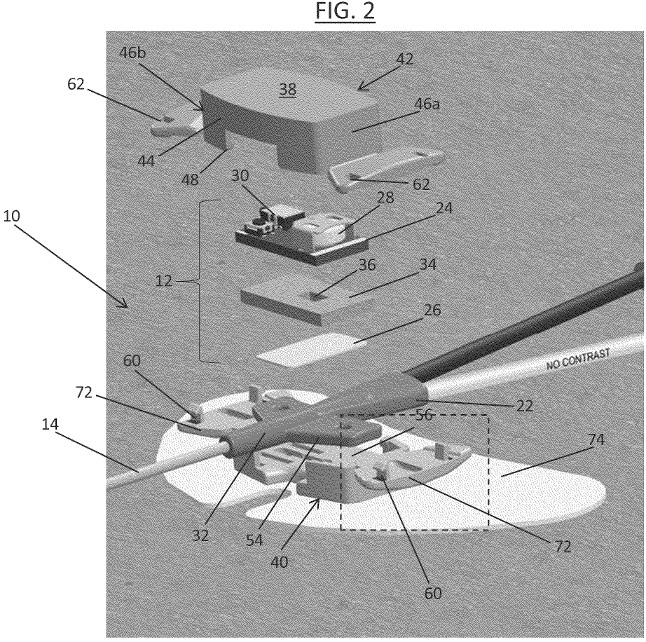

The acoustic wave generator may be provided in a vascular access catheter stabilization device 10, shown in FIGS. 1 and 2, that incorporates an acoustic wave generator 12 for transmitting acoustic waves to a vascular access catheter 14 while the vascular access catheter 14 is inserted in a patient’s blood vessel, thereby generating mechanical vibration in the walls of the vascular access catheter 14.

The catheter stabilization device 10 comprises a hub engagement portion 18 and a base 74.

The hub engagement portion 18 comprises a housing 20 enclosing an acoustic wave generator 12 and adapted to additionally surround at least a portion of the hub 22 of a vascular access catheter 14.

The acoustic wave generator 12 comprises a driver 24 and a piezoelectric plate 26. The driver 24 comprises a power source 28, for example, a battery, and a control unit 30, for example, a printed circuit board. The piezoelectric plate 26 is electrically connected to the driver 24. The driver 24 generates an electric current having alternating polarity that is supplied to a plurality of regions of the piezoelectric plate 26. The alternating polarity of the electric current changes the direction of the electric field between adjacent regions of the piezoelectric plate 26, creating alternating regions of tensile and compressive strain between the regions and producing a surface acoustic wave.

The piezoelectric plate 26 is positioned within the housing 20 such that contact between the piezoelectric plate 26 and the vascular access catheter hub 22 is sufficient to allow transmission of acoustic waves generated in the piezoelectric plate 26 to the vascular access catheter hub 22. This contact allows the acoustic waves generated in the piezoelectric plate 26 to be transmitted from the piezoelectric plate 26 to the vascular access catheter hub 22 as mechanical vibrations which are then transmitted from the vascular access catheter hub 22 to the vascular access catheter 14. The piezoelectric plate 26 has a size and shape corresponding to an upper surface of at least a section of the portion of the vascular access catheter hub 22 enclosed within the housing 20. For example, as shown in FIG. 2, the piezoelectric plate 26 may have a substantially rectangular shape corresponding to the shaft portion 32 of the vascular access catheter hub 22 enclosed with the housing 20.

A compressible cushioning spacer 34 may be included in the housing 20. The cushioning spacer 34 acts to fill any gaps within the housing 20 and assure that the piezoelectric plate 26 positively contacts the vascular access catheter hub 22. The cushioning spacer 34 may be made of any suitable compressible material, for example, elastomeric materials, and compressible foam or elastomers.

The cushioning spacer 34 may be positioned between the driver 24 and the piezoelectric plate 26 as shown in FIG. 2. When the cushioning spacer 34 is positioned between the driver 24 and the piezoelectric plate 26, a via hole 36 is provided in the cushioning spacer 34 to allow the driver 24 to be electrically connected to the piezoelectric plate 26. Alternatively, the cushioning spacer 34 may be positioned between the housing 20 and the driver 24.

The housing 20 comprises a top wall 38, a bottom wall 40, a proximal wall 42, a distal wall 44, and two side walls 46a, 46b. The proximal wall 42 and the distal wall 44 each include an opening 48a, 48b to allow the vascular access catheter 14 to pass through the housing 20 when the vascular access catheter hub 22 is at least partially surrounded by the housing 20.

The driver 24, the piezoelectric plate 26, and the cushioning spacer 34 are contained in an upper portion of the housing 20 between the top wall 38 and the vascular access catheter hub 22. The bottom wall 40 may include a recess 50 that is sized and shaped to receive at least a portion of the vascular access catheter hub 22. The recess 50 may include areas that receive wings 54 extending from the shaft portion 32 of the vascular access catheter hub 22 and surround the edges of the wings 54 to stabilize the vascular access catheter hub 22 within the housing 20.

The housing 20 may have two parts. The first part 56 includes the top wall 38, and the second part 58 includes the bottom wall 40. Any or all of the proximal wall 42, distal wall 44, and side walls 46a, 46b may extend completely from the top wall 38 of the first part 56 or completely from the bottom wall 40 of the second part 58, or may be formed from two portions, one portion extending from the first part 56 and one portion extending from the second part 58. The first part 56 may contain the driver 24, the piezoelectric plate 26, and the cushioning spacer 34. The second part 58 may include the recess 50 that is sized and shaped to receive the vascular access catheter hub 22. Further, the recess 50 may include areas that receive wings 54 extending from the shaft portion 32 of the vascular access catheter hub 22 and surround the edges of the wings 54 to stabilize the vascular access catheter hub 22 within the housing 20.

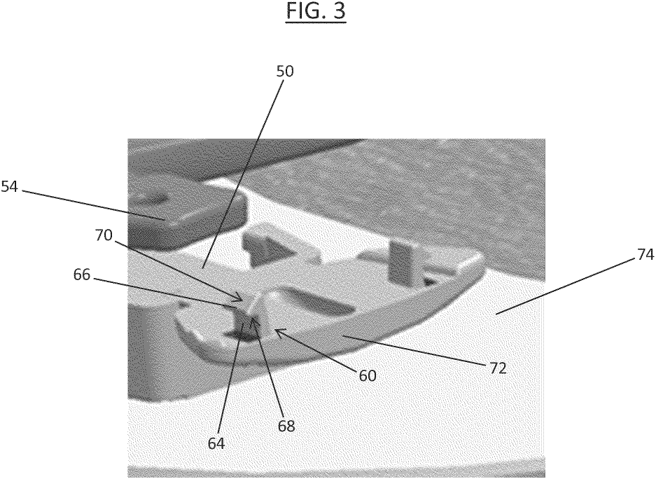

The first part 56 of the housing 20 may be removably attached to or may be locked to the second part 58 by any suitable connection, for example, a connector or an adhesive pad. As shown in FIGS. 1-3, the connection between the first part 56 of the housing 20 and the second part 58 of the housing 20 comprises one or more protrusions 60 on the first part 56 of the housing 20 or the second part 58 of the housing 20 that are received within corresponding openings 62 in the other of the first part 56 of the housing 20 and the second part 58 of the housing 20. In the embodiment shown in FIGS. 1-3, the protrusions 60 are positioned on the second part 58 of the housing 20, and the corresponding openings 62 are positioned on the first part 56 of the housing 20.

The protrusion 60 may comprise a flexible beam 64 attached to the first part 56 of the housing 20 or the second part 58 of the housing 20 at one end and, at the other end, having a tab 66 having a bottom surface 68 extending substantially perpendicularly from the flexible beam 64 and an angled top surface 70. The other of the first part 56 of the housing 20 and the second part 58 of the housing 20 has a corresponding opening 62 adapted to receive the protrusion 60. When the first part 56 of the housing 20 is engaged with the second part 58 of the housing 20, the edge of the opening 62 engages the angled top end 70 of the tab 66 of the protrusion 60 biasing the flexible beam 64 of the protrusion 60 and allowing the tab 66 of the protrusion 60 to pass through the opening 62. Once the tab 66 of the protrusion 60 has passed through the opening 62, the force on the flexible beam 64 is released, the flexible beam 64 returns to its unbiased position, and the substantially perpendicular bottom surface 68 of the tab 66 of the protrusion 60 engages the housing 20 to lock the first part 56 of the housing 20 to the second part 58 of the housing 20. The locking engagement can be disengaged by pressing the tab 66 to bias the flexible beam 64 and allow the tab 66 to pass back through the opening 62.

Flanges 72 may extend substantially perpendicularly from each of the side walls 46a, 46b of each of the first part 56 of the housing 20 and the second part 58 of the housing 20. The connection between the first part 56 of the housing 20 and the second part 58 of the housing 20 may be positioned on the flanges 72. As shown in FIGS. 1 and 2, the protrusions 60 and the corresponding openings 62 of the connection are positioned on the flanges 72 of first part 56 of the housing 20 and the second part 58 of the housing 20.

The vascular access catheter stabilization device 10 further comprises a base 74 attached to the bottom wall 40 of the housing 20. The base 74 has a shape extending beyond the outer perimeter of the housing 20 and may be made of any material that is suitable for removably attaching the vascular access catheter stabilization device 10 to the patient’s skin 16, for example, an adhesive pad, or other elastomer. In use, the base 74 is attached to the patient’s skin 16 near the insertion site 76. The vascular access catheter 14 exits the patient’s skin 16 and passes through the housing 20 of the vascular access catheter stabilization device 10, thereby stabilizing and securing the vascular access catheter 14 to avoid movement of the vascular access catheter 14 within the blood vessel and prevent the vascular access catheter 14 from being pulled from the blood vessel.

The driver 24 may include an adjustment system to adjust the frequency of the electrical current applied to the piezoelectric plate 26 such that the frequency of the acoustic waves may be adjusted. The acoustic waves may have a constant frequency or the frequency may be varied. The frequency of the acoustic waves may be in the low frequency ultrasonic range of 10-60 kHz, or may be in the higher frequency ultrasound range of 90 Hz or more.

The acoustic wave generator 12 may have control features extending through the housing 20. For example, a switch extending through the housing 20 to allow the acoustic wave generator 12 to be turned on and off or a knob or lever for adjusting the frequency of the acoustic waves transmitted to the vascular access catheter hub 22 by the piezoelectric plate 26. Alternatively, the driver 24 may include a receiver to allow the driver 24 to be controlled remotely.

In use, after insertion of the vascular access catheter 14 into the blood vessel of the patient, the vascular access catheter hub 22 is placed in the recess 50 provided in the second part 58 of the housing 20 of the vascular access catheter stabilization device 10 which has been secured by the base 74 to the patient’s skin 16. Then, the first part 56 of the housing 20 of the vascular access catheter stabilization device 10 is placed over the vascular access catheter hub 22 and attached to the second part 58 of the housing 20 of the vascular access catheter stabilization device 10. In this manner, the vascular access catheter stabilization device 10 may be provided during initial insertion of the vascular access catheter 14 into the blood vessel or may be provided for an existing indwelling vascular access catheter 14.

The acoustic wave generator 12 is activated and acoustic waves are transmitted from the acoustic wave generator 12 to the vascular access catheter hub 22 and from the vascular access catheter hub 22 to the vascular access catheter 14 inserted in the patient’s blood vessel resulting in mechanical vibration of the walls of the vascular access catheter 14.

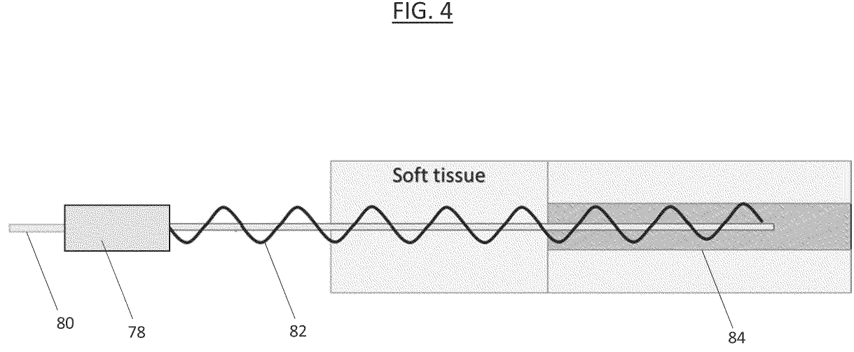

In alternative embodiment (FIG. 4), the acoustic wave generator 78 is provided as a separate device that is attached to the hub of the vascular access catheter or to the catheter tube 80 of the vascular access device. The acoustic wave generator 78 may have all of the features described above with respect to acoustic wave generator 12 the vascular access catheter stabilization device 10. The acoustic wave generator 78 may be attached to the vascular access catheter via any suitable attachment that provides sufficient contact between the acoustic wave generator 78 and the vascular access catheter to allow the acoustic waves 82 to be transmitted to the catheter tube 80 and create mechanical vibrations in the catheter tube 80. For example, the acoustic wave generator 78 may be clipped or clamped to the hub of the vascular access catheter or to the catheter tube 80 of the vascular access device. The clip may comprise arms extending from a housing of the acoustic wave generator 78 that form a snap fit between the acoustic wave generator 78 and hub of the vascular access catheter or the catheter tube 80. Alternatively, the clip may include arms that pivot from an open positon that allows the hub of the vascular access catheter or the catheter tube 80 to be inserted in the clip to a closed positon in which the clip is attached to the hub of the vascular access catheter or the catheter tube 80.In use, the vascular access catheter is inserted into the blood vessel 84 of the patient, and the acoustic wave generator 78 is attached to the vascular access catheter. The acoustic wave generator 78 is activated and acoustic waves are transmitted from the acoustic wave generator 78 to the vascular access catheter hub and then to the catheter tube 80 or directly to the catheter tube 80 resulting in mechanical vibration of the walls of the catheter tube 80 which reduce or prevent the formation and/or buildup of microbial biofilms on the walls of the catheter tube 80, thereby preventing infections.

In a further alternative embodiment, the acoustic wave generator is incorporated into a vascular access catheter. The acoustic wave generator may have all of the features described above with respect to the vascular access catheter stabilization device 10. The vascular access catheter comprises a catheter tube, a hub comprising a housing through which the vascular access catheter tube passes, and an acoustic wave generator enclosed within the housing. A cushioning spacer as described above with respect to the vascular access catheter stabilization device 10 may be included in the housing. The acoustic waves generated by the acoustic wave generator may be transmitted to the catheter tube via the hub or directly to the catheter tube passing through the hub. Flanges may extend from opposite sides of the housing to act as the wings of the vascular access catheter.

In use, the vascular access catheter is inserted into the blood vessel of the patient, the acoustic wave generator is activated and acoustic waves are transmitted from the acoustic wave generator to the vascular access catheter hub and then to the catheter tube or directly to the catheter tube resulting in mechanical vibration of the walls of the catheter tube.

With the inventive vascular access catheter stabilization device and the inventive vascular access catheter, acoustic waves may be propagated as mechanical vibration along the entire length of the vascular access catheter to prevent microbial biofilm formation, and if a microbial biofilm has already formed on the walls of the vascular access catheter, break up the microbial biofilm, thereby preventing infection. The acoustic waves may be applied intermittently or during the entire indwelling time.

Unlike prior art solutions for removing microbial biofilms using cleaning processes or inhibiting the formation of microbial biofilms by killing the microbes via disinfecting caps, thereby preventing microbes from entering the vascular access catheter, the present inventions inhibit microbes from attaching to the walls of the vascular access catheter, thereby suppressing the formation of a microbial biofilm. Further, the mechanical vibration created by the acoustic waves accomplishes the reduction in the formation and growth of microbial biofilms and the removal of microbial biofilms without the use of any cleaning solutions and without pulling the vascular access catheter from the patient. This protection can be provided for potentially as long as the vascular access catheter remains suitable for use.

Whereas particular aspects of this invention have been described above for purposes of illustration, it will be evident to those skilled in the art that numerous variations of the details of the present invention may be made without departing from the invention.

The invention claimed is:

1. A vascular access catheter stabilization device comprising:
a hub engagement portion comprising a housing enclosing an acoustic wave generator and adapted to additionally surround at least a portion of a hub of a vascular access catheter; and
a base adapted for removable attachment to a patient’s skin.

2. The vascular access catheter stabilization device of claim 1, wherein the acoustic wave generator comprises a driver electrically connected to a piezoelectric plate.

3. The vascular access catheter stabilization device of claim 2, wherein the driver comprises a power source for supplying electric current and a control unit for controlling the transmission of the electric current to piezoelectric plate.

4. The vascular access catheter stabilization device of claim 2, wherein the piezoelectric plate is positioned within the housing to be in contact with the hub of the vascular access catheter when the hub of the vascular access catheter is enclosed in the housing.

5. The vascular access catheter stabilization device of claim 2, wherein the piezoelectric plate has a size and shape corresponding to an upper surface of at least a section of a portion of the hub of the vascular access catheter enclosed within the housing, and contact between the piezoelectric plate and the hub of the vascular access catheter is sufficient to allow transmission of acoustic waves generated in the piezoelectric plate to the hub of the vascular access catheter.

6. The vascular access catheter stabilization device of claim 2, further comprising a compressible cushioning spacer that acts to fill any gaps within the housing and assure that the piezoelectric plate positively contacts the hub of the vascular access catheter.

7. The vascular access catheter stabilization device of claim 2, further comprising a compressible cushioning spacer that acts to fill any gaps within the housing and assure that the piezoelectric plate positively contacts the hub of the vascular access catheter, wherein the cushioning spacer is positioned between the driver and the piezoelectric plate.

8. The vascular access catheter stabilization device of claim 1, wherein the housing comprises a top wall, a bottom wall, a proximal wall, a distal wall, and two side walls, and the proximal wall and the distal wall each have an opening to allow the vascular access catheter to pass through the housing when the hub of the vascular access catheter is at least partially surrounded by the housing.

9. The vascular access catheter stabilization device of claim 8, wherein the driver, the piezoelectric plate, and the cushioning spacer are contained in an upper portion of the housing between the top wall and the hub of the vascular access catheter.

10. The vascular access catheter stabilization device of claim 8, wherein the bottom wall has a recess that is sized and shaped to receive at least a portion of the hub of the vascular access catheter.

11. The vascular access catheter stabilization device of claim 10, wherein the recess comprises areas that receive wings extending from a shaft portion of the hub of the vascular access catheter and surround edges of the wings to stabilize the hub of the vascular access catheter within the housing.

12. The vascular access catheter stabilization device of claim 8, wherein the housing comprises a first part including the top wall and a second part including the bottom wall, and the first part of the housing is removeably connected to or locked to the second part of the housing.

13. The vascular access catheter stabilization device of claim 12, wherein a connection between the first part of the housing and the second part of the housing comprises one or more protrusions on the first part of the housing or the second part of the housing that are received within corresponding openings in the other of the first part of the housing and the second part of the housing.

14. The vascular access catheter stabilization device of claim 13, wherein the one or more protrusions comprise a flexible beam attached to the housing at one end and, at the other end, has a tab having a bottom surface extending substantially perpendicularly from the flexible beam and an angled top surface.

15. The vascular access catheter stabilization device of claim 12, wherein flanges extend substantially perpendicularly from each side wall of each of the first part of the housing and the second part of the housing and the connection or locking of the first part of the housing to the second part of the housing is provided via the flanges.

16. The vascular access catheter stabilization device of claim 1, wherein the base is an adhesive pad.

17. The vascular access catheter stabilization device of claim 1, wherein the acoustic waves generated by the acoustic wave generator have a frequency in the ultrasonic range above 20 kHz.

18. A method of preventing infections in vascular access devices, the method comprising:
attaching an acoustic wave generator to the vascular access catheter;
activating the acoustic wave generator to produce acoustic waves; and
transmitting the acoustic waves to the vascular access catheter to create mechanical vibrations in the vascular access catheter.

19. The method of claim 18, wherein the acoustic waves generated by the acoustic wave generator have a frequency in the ultrasonic range.

20. The method of claim 18, wherein the acoustic wave generator is attached to a catheter tube of the vascular access catheter.

21. The method of claim 18, wherein the acoustic wave generator is attached to a hub of the vascular access catheter.

22. The method of claim 18, wherein the acoustic wave generator has a contact area having a size and shape corresponding to a surface of the vascular access catheter, and contact between the acoustic wave generator and the vascular access catheter is sufficient to allow transmission of the acoustic waves generated by the acoustic wave generator to the vascular access catheter.

23. The method of claim 18, wherein the acoustic wave generator is a separate device that is removably clipped to the vascular access catheter.
